(19) United States
(12) Patent Application Publication
(10) Pub. No.: US 2005/0009848 A1
(43) Pub. Date: Jan. 13, 2005

(54) Use of antivirals against inflammatory bowel diseases

(75) Inventor: Victor Brantl, Schliengen (DE)

Correspondence Address: HOGAN & HARTSON L.L.P., 500 S. GRAND AVENUE, SUITE 1900, LOS ANGELES, CA, 90071-2611, US

(73) Assignee: ICN Pharmaceuticals Switzerland Ltd.

(21) Appl. No.: 10/618,176

(22) Filed: Jul. 10, 2003

(51) Int. Cl. A61K 31/522; A61K 31/4745

(52) U.S. Cl. 514/263.36; 514/291

(57) ABSTRACT

A method for the prophylaxis or treatment of an inflammatory bowel disease is provided, comprising administering to a patient having or at risk of developing an inflammatory bowel disease a therapeutically or preventatively effective amount of one or more antivirals selected from the group consisting of an antiviral active against herpes viruses, pharmaceutically acceptable salts thereof, and mixtures thereof, excluding anti-inflammatory agents selected from the group consisting of salicylates and salicylates prodrugs. The antiviral may be used in combination with additional active agents effective against inflammatory bowel disease.

### BACKGROUND OF INVENTION

1. Field of the Invention

This invention relates to a method for treatment and prevention of inflammatory bowel disease which comprises administering to a subject an effective amount of at least one anti-viral active against Herpes viruses, sub-types of Herpes viruses, and/or cytomegaly, or a pharmaceutically acceptable salt thereof, or mixtures thereof. This invention further relates to the use of such compounds in the preparation of a medicament for the treatment and prevention of inflammatory bowel disease.

2. Background of the Invention

Inflammatory bowel disease (IBD) is a term used in the art to generically encompass diseases of the intestine such as ulcerative colitis (UC), irritable bowel syndrome, irritable colon syndrome and Crohn's disease (CD). For many of these diseases, in particular CD, the origin of the disease (bacterial, viral or autoimmune) is unknown. There is sufficient overlap in the diagnostic criteria for UC and CD that it is sometimes impossible to say which a given patient has; however, the type of lesion typically seen is different, as is the localization. UC mostly appears in the colon, proximal to the rectum, and the characteristic lesion is a superficial ulcer of the mucosa; CD can appear anywhere in the bowel, with occasional involvement of stomach, esophagus and duodenum, and the lesions are usually described as extensive linear fissures. IBD is rather common, with a prevalence that is claimed to be in the range of 70-170 in a population of 100,000.

Crohn's disease is currently neither medically nor surgically curable, requiring approaches to treatment that maintains symptomatic control, quality of life, and minimizes short- and long-term toxicity of therapy. The current therapy of IBD usually involves the administration of anti-inflammatory or immunosuppressive agents, such as sulfasalazine, corticosteroids, 6-mercaptopurine/azathioprine, or cyclosporine, which usually bring only partial results. For example, IBD's such as Crohn's disease or ulcerative colitis have been treated in the past with salicylic acid derivatives (such as 5-aminosalicylic acid, also known as 5-ASA or mesalazine; and prodrugs thereof, such as sulfasalazine). Possible side effects of 5-ASA preparations include nausea, vomiting, heartburn, diarrhea and headaches. Other treatments have been based on corticosteroids such as cortisone, however prolonged use of steroids has been known to result in side effects such as weight gain, shrinking of the adrenal glands, gray cataract, glaucoma, osteoporosis and diabetes mellitus. The use of immune modifying drugs such as 6-mercaptopurine and its prodrug azathioprine against Crohn's disease has increased in recent years, but these drugs are slow acting and clinical activity cannot be expected until several weeks or even months of treatment has elapsed. In recent years the use of immunomodulating monoclonal antibodies that neutralize TNF-α has been contemplated, the only example of such an antibody that obtained marketing approval for use against Crohn's disease currently being infliximab. A drawback of this therapy is the high risk of severe infections when administered by injection and the risk of lymphoproliferative disease. A reported side effect of the treatment with infliximab is bilateral anterior toxic optic neuropathy.

If anti-inflammatory and/or immunosuppressive therapies fail, colectomies are the last line of defense. About 30% of CD patients will need surgery within the first year after diagnosis. In subsequent years, the rate is about 5% per year. Unfortunately, CD is characterized by a high rate of recurrence; about 5% of patients need a second surgery each year after initial surgery. In UC, a further reason for resorting to surgery is that the patients are known to be at much increased risk for developing colorectal cancer, starting 10-15 years after the diagnosis of ulcerative colitis. Presumably this is due to the recurrent cycles of injury to the epithelium, followed by regrowth, increasing the risk of transformation. Accordingly, colostomy is used as prophylaxis against the development of cancer in UC patients.

Rüther et al., (Deutsche medizinische Wochenzeitschrift, 117: 46-50 (1992) found that in a specific patient with Crohn's disease, in which HSV-I and HSV-II DNA was detected in the intestinal mucosa, the co-use of acyclovir with the classical mesalazin-corticosteroid anti-Crohn's disease therapy “changed the morphological and clinical findings decisively”, and the authors concluded that “if a virus infection is found to be a pathogenic co-factor in a case of Crohn's disease, a therapeutic trial with a suitable antiviral agent seems reasonable”. This publication thus still used conventional treatment against Crohn's disease.

From the above it is evident that there still exists the need of drugs and therapies that are effective against inflammatory bowel diseases and that avoid the disadvantages of the prior art drugs and treatment.

### SUMMARY OF THE INVENTION

One aspect of the present invention provides a method for the prophylaxis or treatment of inflammatory bowel disease in a patient having or at risk of developing an inflammatory bowel disease, wherein the method comprises providing at least one antiviral selected from the group consisting of antivirals active against Herpes viruses and pharmaceutically acceptable salts thereof; and administering the antiviral to the subject in an amount effective to treat or prevent the inflammatory bowel disease, with the proviso that the method excludes administration of an anti-inflammatory agent selected from the group consisting of salicylates and salicylate prodrugs.

The method may further comprise administering the antiviral with one or more additional agents effective against inflammatory bowel disease, wherein the antiviral and the additional active agent are administered simultaneously in admixture, separately and simultaneously, or separately in any order. In this embodiment, compounds possessing a 1-ribofuranosyl-1H-1,2,4-triazole moiety are excluded from additional active agents suitable for purposes of this invention.

The invention thus provides a method of treating or preventing an inflammatory bowel disease irrespective of whether a viral infection is a pathogenic cofactor or not. It is to be understood that according to the invention the antiviral active against Herpes viruses or a pharmaceutically acceptable salt thereof is used as the actual active agent against inflammatory bowel disease, not merely as an antiviral co-adjuvant in an otherwise conventional treatment or prevention of the inflammatory bowel disease.

This invention further provides a medicament for the treatment or prophylaxis of an inflammatory bowel disease, comprising one or more antivirals in combination with one or more agents effective against an inflammatory bowel disease for administration to a mammal simultaneously in admixture, separately and concomitantly, or successively, wherein said antiviral is in an amount effective to treat or prevent said inflammatory disease and is selected from the group consisting of antivirals active against Herpes viruses and pharmaceutically acceptable salts thereof, with the proviso that the medicament excludes anti-inflammatory agents selected from the group consisting of salicylates and salicylate prodrugs.

This invention further provides the use of at least one antiviral alone or in combination with one or more additional active agents against inflammatory bowel disease in the preparation of a medicament against an inflammatory bowel disease, wherein the antiviral is selected from the group consisting of antivirals active against Herpes viruses and pharmaceutically acceptable salts thereof, with the proviso that the use excludes the use of anti-inflammatory agents selected from the group consisting of salicylates and salicylate prodrugs.

Additional advantages and novel features of this invention shall be set forth in part in the description that follows, and in part will become apparent to those skilled in the art upon examination of the following specification or may be learned by the practice of the invention. The advantages of the invention may be realized and attained by means of the instrumentalities, combinations, compositions, and methods particularly pointed out in the appended claims.

### DETAILED DESCRIPTION OF THE INVENTION

One aspect of the present invention provides a method for the treatment or prophylaxis of an inflammatory bowel disease, comprising providing at least one antiviral selected from the group consisting of antivirals active against Herpes viruses and pharmaceutically acceptable salts thereof, and administering to a subject in need thereof the antiviral in an amount or dose range effective to treat or prevent the inflammatory bowel disease, with the proviso that the method excludes administration of anti-inflammatory agents selected from the group consisting of salicylates and salicylate prodrugs.

The term “inflammatory bowel disease” as used herein includes all forms of inflammatory processes in the gastrointestinal tissue, including but not limited to, pseudomembranous colitis, hemorrhagic colitis, hemolytic-uremic syndrome colitis, collagenous colitis, ischemic colitis, radiation colitis, drug and chemically induced colitis, diversion colitis, ulcerative colitis, irritable bowel syndrome, irritable colon syndrome and Crohn's disease; and within Crohn's disease all the subtypes including active, refractory, and fistulizing and Crohn's disease.

The term “against inflammatory bowel disease” as used herein refers to a therapeutic treatment or prophylaxis for inflammatory bowel disease, and an active agent “effective against inflammatory bowel disease” refers to an agent serves in the treatment or prophylaxis of inflammatory bowel disease.

An “effective amount” is intended to mean that amount of compound that, when administered to a mammal in need of treatment or prophylaxis, is sufficient to effect treatment or prevention, respectively, of inflammatory bowel disease.

The term “treating” is intended to mean at least the mitigation of inflammatory bowel disease in a mammal, such as a human, that is affected, at least in part, by the disease, and includes, but is not limited to, modulating and/or inhibiting the disease condition; and/or alleviating the disease condition.

The term “prophylaxis” is intended to mean at least preventing the disease condition from occurring in a mammal, particularly when the mammal is found to be predisposed to having the disease condition but has not yet been diagnosed as having it.

The term “Herpes viruses” includes, but is not limited to, alpha-Herpes viruses such as Herpes simplex virus type 1 (HSV-1), Herpes simplex virus type 2 (HSV-2) and varicella-zoster virus (VZV); beta-Herpes viruses such as cytomegaly virus (CMV); and gamma-Herpes viruses such as Epstein-Barr virus (EBV).

The terms “patient” and “subject” as used herein include any animal, including mammals and humans.

This invention further provides a medicament against an inflammatory bowel disease, comprising one or more antivirals in combination with one or more agents effective against an inflammatory bowel disease for administration to a mammal simultaneously in admixture, separately and concomitantly, or successively, wherein said antiviral is in an amount effective to treat or prevent said inflammatory disease and is selected from the group consisting of antivirals active against Herpes viruses and pharmaceutically acceptable salts thereof, with the proviso that the medicament excludes anti-inflammatory agents selected from the group consisting of salicylates and salicylate prodrugs. In one embodiment, the medicament comprises an antiviral active against a Herpes virus in an amount between about 200 mg and 5 grams.

The term “medicament” as used herein includes any type of medicament for oral, nasal, topical, transdermal, rectal and parenteral administration (e.g., administration by injection), whereby the medicament can be a single dosage containing at least one antiviral selected from the group consisting of antivirals active against Herpes viruses, and/or pharmaceutically acceptable salts thereof, and mixtures thereof, alone or in admixture with at least one additional agent effective against inflammatory bowel disease, or alternatively at least one antiviral selected from the group consisting of antivirals active against Herpes viruses and pharmaceutically acceptable salts thereof, and mixtures thereof and at least on additional agent effective against inflammatory bowel disease in separate dosage forms. Additionally, the term “medicament” includes a kit with one or more dosage forms containing at least one antiviral selected from the group consisting of antivirals active against Herpes viruses and/or pharmaceutically acceptable salts thereof, and separately at least one dosage form containing at least one additional agent effective against inflammatory bowel disease, or a kit with one or more dosage forms containing at least one antiviral selected from the group consisting of antivirals active against Herpes viruses and pharmaceutically acceptable salts thereof, alone or in admixture with one or more additional agents effective against inflammatory bowel disease and one or more separate dosage forms containing either one or more antivirals selected from the group consisting of antivirals active against Herpes viruses and pharmaceutically acceptable salts thereof, or an additional agent effective against inflammatory bowel disease.

An antiviral active against a Herpes virus suitable for purposes of this invention includes, but is not limited to, acyclovir, brivudine, cidofovir, famciclovir, foscarnet, ganciclovir, penciclovir, trifluridine, valacyclovir, valgancyclovir, and pharmaceutically acceptable salts thereof and mixtures thereof. These antivirals are known compounds and are given by their international nonproprietary names.

The term “pharmaceutically acceptable acid addition salt” refers to a compound obtained upon treatment of a compound (e.g., an antiviral active against a Herpes virus, or any other compound to be administered according to this invention) with a pharmaceutically acceptable acid including, but not limited to, hydrochloric, hydrobromic, acetic, propionic, p-toluenesulfonic, sulfuric, nitric or lactic acid. Methods of preparing acid addition salts of such compounds are well known to those skilled in the art. One example of such a method is described in U.S. Pat. No. 6,455,408 B1, which is incorporated herein by reference.

When an acid addition salt of a compound is intended for administration by injection according to this invention, the amount of acid added to the compound in the preparation of the salt may be restricted by the pH of the aqueous solution of the resulting acid addition salt, which should be within physiologically tolerable ranges.

In certain cases, where the composition or medicament contains an additional active agent against inflammatory bowel disease that contains an acidic hydrogen (e.g., acyclovir, valacyclovir, ganciclovir, valgancyclovir, and pencyclovir), a “pharmaceutically acceptable salt” of the additional agents may be formed by deprotonation of the acidic hydrogen. Such deprotonation salts include, for example, of the sodium or potassium salts of acyclovir and valacylovir are obtained by deprotonation of the 1-imino hydrogen. Other methods for preparing salts of agents containing an acidic hydrogen are well known in the art.

One embodiment of this invention comprises a method of treating or preventing inflammatory bowel disease in a patient, comprising administering to a subject in need of such treatment or prophylaxis a therapeutically effective or preventatively effective amount of at least one antiviral selected from the group consisting of antivirals active against Herpes viruses and acid addition salts thereof. The amount of the antiviral that is effective for the treatment or prevention of an inflammatory bowel disease will vary depending on the compound used and on other factors, such as the body weight of the subject, and can be determined by clinical studies on laboratory animals or on human volunteers. One indication that a therapeutically effective in vivo amount has been administered is the induction of a clinical remission of the inflammatory bowel disease in question. It is well within the ordinary skill of the art to modify the route of administration and dosage regimen in order to manage the pharmacokinetics of the present compounds for maximum beneficial effect in patients.

A proviso of the method of this invention is that the method excludes the administration of an anti-inflammatory agent selected from the group consisting of salicylates and the prodrugs thereof. Examples of such salicylates and prodrugs include mesalazine and sulfasalazine, respectively.

An exemplary dosing regime for oral treatment or prophylaxis of inflammatory bowel disease comprises orally administering at least one antiviral selected from the group consisting of antivirals active against Herpes viruses and acid addition salts thereof in a dose range of about 200 mg to 5 g of the antiviral(s) per day over a period of 1 to 4 weeks. With the lower doses within this range the treatment may be extended to up to twelve months, in particular for prophylactic treatment.

A further exemplary dosing regime for oral treatment or prophylaxis of inflammatory bowel disease comprises orally administering to a subject for a period between about 1 week to 12 months (a) the antiviral in a dose between about 200 mg to about 5 g per day and (b) an additional agent in an amount that is between about half the dosage and the same dosage of the additional agent which, when administered alone, is effective to treat or prevent the inflammatory bowel disease.

As an example, a method can comprise orally administering about 250 mg of an antiviral active against a Herpes virus (e.g., famciclovir) and about 3 mg of the additional agent (e.g., budesonide). In this embodiment, the antiviral and the additional agent can be formulated together or can be administered as separated formulations. When the additional agent is budesonide, it is preferably formulated with an enteric coating.

A further example of an oral dosing regime comprises orally administering to the subject for a period between about 1 to 4 weeks (a) the antiviral in a dose between about 200 mg to about 5 g per day and (b) the additional agent in an amount that is between about half the dosage and the same dosage which, when the additional agent is administered alone, is effective to treat or prevent said inflammatory bowel disease.

An exemplary dosage regime for intravenous treatment or prophylaxis of inflammatory bowel disease comprises administering at least one antiviral selected from the group consisting of antivirals active against Herpes viruses and acid addition salts thereof comprises intravenous administration of an intravenous solution comprising about 5 to 15 mg of the antiviral(s) per kilogram of body weight of the patient over a period of about 60 minutes, in intervals of about 6 to about 8 hours, for 7 to 10 days.

Since antivirals active against Herpes viruses and the pharmaceutically acceptable salts thereof that are suitable for purposes of this invention have been used for other purposes for several years, many dosage forms and routes of administration are known, and therefore all appropriate dosage forms and routes of administration may be utilized in the methods of this invention. For example, in addition to oral administration, the antiviral may given intravenously, intramuscularly, intraperitoneally, topically, and the like, all of which are known.

Another embodiment of this invention comprises a method of treatment or prophylaxis of an inflammatory bowel disease in a subject in need of said treatment or prophylaxis, said method comprising (i) providing at least one antiviral selected from the group consisting of antivirals active against Herpes viruses and pharmaceutically acceptable salts thereof; (ii) providing at least one additional agent effective against an inflammatory bowel disease, and (iii) administering the antiviral(s) and the additional agent(s) to the subject in an amount effective to treat or prevent the inflammatory bowel disease, with the proviso that the method excludes administration of an anti-inflammatory agent selected from the group consisting of salicylates and salicylate prodrugs. In this embodiment, the antiviral(s) and the additional agent(s) can be administered to the subject simultaneously as an admixture, separately and concomitantly, or successively

An antiviral active against a Herpes virus or a pharmaceutically acceptable salt thereof, whether alone or in the combination therapies or preventions as discussed herein, may be administered in any appropriate pharmaceutical formulation, and under any appropriate protocol. Thus, administration may take place by various routes including oral (for example as tablets, microbeads, lozenges, hard or soft capsules, solutions, aqueous or oily suspensions, emulsions, dispersible powders or granules, syrups or elixirs), parenteral (including subcutaneous injections, intravenous, intramuscular, by intrastemal injection or infusion techniques), transdermal (for example as a patch which may include a penetration enhancement agent), by inhalation spray (such as in the form of a finely divided powder with an appropriate powdery diluent or a liquid aerosol, to form an ordered mixture that can be inhaled with a dry powder inhaler; or as an aerosolizable solution, to be inhaled e.g. with a metered dose inhaler0, for administration by insufflation (for example as a finely divided powder), for topical use (for example as creams, ointments, gels, or aqueous or oily solutions or suspensions), buccal and suppository administration, and other routes of administration, and in dosage unit formulations containing conventional non-toxic pharmaceutically acceptable carriers, adjuvants, and vehicles.

Suitable pharmaceutically-acceptable excipients for a tablet formulation include, for example, inert diluents such as lactose, sodium carbonate, calcium phosphate or calcium carbonate, granulating and disintegrating agents such as corn starch or algenic acid; binding agents such as starch; lubricating agents such as magnesium stearate, stearic acid or talc; preservative agents such as ethyl or propyl p-hydroxybenzoate, and anti-oxidants, such as ascorbic acid. Tablet formulations may be uncoated or coated either to modify their disintegration and the subsequent absorption of the active ingredient within the gastrointestinal tract, or to improve their stability and/or appearance, in either case, using conventional coating agents and procedures well known in the art.

Compositions for oral use may be in the form of hard gelatin capsules in which the active ingredient is mixed with an inert solid diluent, for example, calcium carbonate, calcium phosphate or kaolin, or as soft gelatin capsules in which the active ingredient is mixed with water or an oil such as peanut oil, liquid paraffin, or olive oil.

Aqueous suspensions generally contain the active ingredient in finely powdered form together with one or more suspending agents, such as sodium carboxymethylcellulose. methylcellulose, hydroxypropylmethylcellulose, sodium alginate, polyvinyl-pyrrolidone, gum tragacanth and gum acacia; dispersing or wetting agents such as lecithin or condensation products of an alkylene oxide with fatty acids (for example polyoxethylene stearate), or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives (such as ethyl or propyl p-hydroxybenzoate, anti-oxidants (such as ascorbic acid), coloring agents, flavoring agents, and/or sweetening agents (such as sucrose, saccharine or aspartame).

Oily suspensions may be formulated by suspending the active ingredient in a vegetable oil (such as arachis oil, olive oil, sesame oil or coconut oil) or in a mineral oil (such as liquid paraffin). The oily suspensions may also contain a thickening agent such as beeswax, hard paraffin or cetyl alcohol. Sweetening agents such as those set out above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water generally contain the active ingredient together with a dispersing or wetting agent, suspending agent and one or more preservatives. Suitable dispersing or wetting agents and suspending agents are exemplified by those already mentioned above.

Additional excipients such as sweetening, flavoring and coloring agents, may also be present.

The pharmaceutical compositions of the invention may also be in the form of oil-in-water emulsions. The oily phase may be a vegetable oil, such as olive oil or arachis oil, or a mineral oil, such as for example liquid paraffin or a mixture of any of these. Suitable emulsifying agents may be, for example, naturally-occurring gums such as gum acacia or gum tragacanth, naturally-occurring phosphatides such as soya bean, lecithin, an esters or partial esters derived from fatty acids and hexitol anhydrides (for example sorbitan monooleate) and condensation products of the said partial esters with ethylene oxide such as polyoxyethylene sorbitan monooleate. The emulsions may also contain sweetening, flavoring and preservative agents.

Syrups and elixirs may be formulated with sweetening agents such as glycerol, propylene glycol, sorbitol, aspartame or sucrose, and may also contain a demulcent, preservative, flavoring and/or coloring agent.

The pharmaceutical compositions may also be in the form of a sterile injectable aqueous or oily suspension, which may be formulated according to known procedures using one or more of the appropriate dispersing or wetting agents and suspending agents, which have been mentioned above. A sterile injectable preparation may also be a sterile injectable solution or suspension in a non-toxic parenterally-acceptable diluent or solvent, for example a solution in 1,3-butanediol.

Suppository formulations may be prepared by mixing the active ingredient with a suitable non-irritating excipient that is solid at ordinary temperatures but liquid at the rectal temperature and will therefore melt in the rectum to release the drug. Suitable excipients include, for example, cocoa butter and polyethylene glycols.

Topical formulations, such as creams, ointments, gels and aqueous or oily solutions or suspensions, may generally be obtained by formulating an active ingredient with a conventional, topically acceptable, vehicle or diluent using conventional procedures well known in the art.

Compositions for administration by insufflation may be in the form of a finely divided powder containing particles of average diameter of, for example, 30 μm or much less, the powder itself comprising either active ingredient alone or diluted with one or more physiologically acceptable carriers such as lactose. The powder for insufflation is then conveniently retained in a capsule containing, for example, 1 to 50 mg of active ingredient for use with a turbo-inhaler device, such as is used for insufflation of the known agent sodium cromoglycate.

Compositions for administration by inhalation may be in the form of a conventional pressurized aerosol arranged to dispense the active ingredient either as an aerosol containing finely divided solid or liquid droplets. Conventional aerosol propellants such as volatile fluorinated hydrocarbons or hydrocarbons may be used and the aerosol device is conveniently arranged to dispense a metered quantity of active ingredient.

Another formulation employed in the methods of this invention comprises transdermal delivery devices, patches, bandages, and the like. Such transdermal patches may be used to provide continuous or discontinuous infusion of the compounds of the present invention in controlled amounts. The construction and use of transdermal patches for the delivery of pharmaceutical agents is well known in the art. See, for example, U.S. Pat. No. 5,023,252, the disclosure of which is herein incorporated by reference. Such patches may be constructed for continuous, pulsatile, or on demand delivery of pharmaceutical. For example, a dose of one or more antivirals selected from the group consisting of antivirals active against Herpes viruses, pharmaceutically acceptable salts thereof, and mixtures thereof may be combined with skin penetration enhancers such as propylene glycol, polyethylene glycol, isopropanol, oleyl alcohol, ethoxydiglycol, sodium xylene sulfonate, ethanol, oleic acid, N-methylpyrrolidone, laurocapram, alkanecarboxylic acids, dimethylsulfoxide, polar lipids, and N-methyl-2-pyrrolidone, and the like, which increase the permeability of the skin to the dose of the antiviral and permit the antiviral to penetrate through the skin and into the bloodstream. A patch comprising one or more antivirals selected from the group consisting of antivirals active against Herpes viruses, pharmaceutically acceptable salts thereof, and mixtures thereof may further comprise one or more agents such as moisturizers, humectants, oils, emulsifiers, thickeners, thinners, surface active agents, fragrances, preservatives, antioxidants, vitamins, or minerals. The antiviral may also be further combined with a polymeric substance, such as ethylcellulose, hydroxypropyl cellulose, ethylene/vinylacetate, polyvinyl pyrrolidone, and the like, to provide the composition in gel form, which may be dissolved in solvent such as methylene chloride, evaporated to the desired viscosity, and then applied to backing material to provide a patch. The backing can be any of the conventional materials such as polyethylene, ethyl-vinyl acetate copolymer, polyurethane and the like. For further information on formulations, see Chapter 25.2 in Volume 5 of Comprehensive Medicinal Chemistry (Corwin Hansch; Chairman of Editorial Board), Pergamon Press 1990, which is specifically incorporated herein by reference.

The administration of one or more antivirals selected from the group consisting of antivirals active against Herpes viruses and pharmaceutically acceptable salts thereof, alone or in combination with another active ingredient as described herein, need not be restricted to a single daily injection, but may include alternative frequencies and routes. For example, where relatively high amounts of the antiviral need to be delivered, two to four or more daily injections are contemplated. Similarly, where high plasma concentrations of the antiviral are desired over an extended period, a permanent delivery method is contemplated. For example, a more permanent delivery may include the use of a continuous infusion, an osmotic pump, or a sustained release implant.

With respect to the dosage of one or more antivirals selected from the group consisting of antivirals active against Herpes viruses and pharmaceutically acceptable salts thereof, whether alone or in combination with one or more additional agents against inflammatory bowel disease, one of ordinary skill in the art will recognize that a therapeutically effective amount will vary with the infection or condition to be treated, its severity, the treatment regimen to be employed, the pharmacokinetics of the agent used, as well as the patient (animal or human) treated. It is further contemplated that while treatment success may be achieved at relatively low plasma concentrations of the antiviral, other conditions may require relatively high dosages.

In embodiments comprising a water-soluble antiviral or a salt thereof active against a Herpes virus the antiviral can be administered in the form of an injectable, especially an intravenous solution in a pharmaceutically acceptable solvent, such as water for injection (WFI) or physiological saline solution, preferably buffered to a pH of about 5.0 to about 7.5, and optionally by using suited pharmaceutically acceptable co-solvents such as ethanol or DMSO. Conventional buffers such as phosphates, bicarbonates or citrates can be used for buffering. These solutions may be prepared immediately prior to use.

As stated, a further embodiment, this invention provides a method of treatment or prophylaxis of inflammatory bowel disease in a patient comprising administering at least one antiviral selected from the group consisting of antivirals active against Herpes viruses, pharmaceutically acceptable salts thereof, and mixtures thereof, in combination with at least one other agent effective against inflammatory bowel disease, with the proviso that the method excludes administration of anti-inflammatory agents selected from the group consisting of salicylates and salicylate prodrugs. Combination therapies according to the present invention comprise at least one antiviral selected from the group consisting of antivirals active against Herpes viruses and pharmaceutically acceptable salts thereof co-formulated and/or co-used with at least one other pharmaceutically active ingredient. In this method, an antiviral may be administered separately or together with another active agent against inflammatory bowel disease, and when administered separately this may occur simultaneously or separately in any order. The dosages of the active agent(s) and antiviral and the relative timings of administration will be selected in order to achieve the desired combined therapeutic effect.

Thus, according to another embodiment of this invention, one or more antivirals selected from the group consisting of antivirals active against Herpes viruses and pharmaceutically acceptable salts thereof can be co-administered and/or co-formulated with at least one additional active agent including, but not limited to:

(a) conventional agents used in the field of treatment of inflammatory bowel diseases including, but not limited to, corticosteroids, mercaptopurine, azathioprine, metothrexate, cyclosporine and tacrolimus. In the case of the corticosteroids, systemically inactive corticosteroids are preferred. The term “systemically inactive” refers to corticosteroids that are degraded systemically to inactive metabolites and that act exclusively or almost exclusively by topical route. These corticosteroids act only topically, i.e., only on the mucosa of the intestine. However, this does not preclude their oral co-administration, as it is possible to prepare oral formulations with a suitable coating that permits release only in the intestine, such as an enteric coating. Methods of providing a compound with an enteric coating are well known to those skilled in the art. Examples of such systemically inactive corticosteroids that can be co-used according to the invention include, but are not limited to, budesonide, fluticasone, and the pharmaceutically acceptable salts thereof, such as fluticasone dipropionate; and/or

(b) further antivirals different from the antiviral active against Herpes viruses and excluding compounds possessing a 1-ribofuranosyl-1H-1,2,4-triazole moiety, wherein the further antivirals include, but are not limited to, abacavir, adefovir, amantadine, amprenavir, atazanavir, capravirine, delavirdine, didanosine, efavirenz, emivirin, emtricitabine, enfurvirtide, fosamprenavir, idoxuridine, indinavir, lamivudine, lopinavir, memantine, mozenavir, nelfinavir, nevirapine, oseltamivir, rimantidine, pentafuside, ritonavir, saquinavir, stavudine, tenofovir, tipranavir, zalcitabine, zanamivir, zidovudine and the pharmaceutically acceptable salts thereof. The term “possessing a 1-ribofuranosyl-1H-1,2,4-triazole moiety” refers to compounds related to D-or L-ribavirin, D-or L-viramidine, and their pharmaceutically acceptable salts; and/or

(c) additional agents active against inflammatory bowel diseases including, but not limited to, nitric oxide releasing steroid derivatives such as NicOx 1015 (NicOx SA); nitric oxide releasing salicylates such as NicOx 456 (NicOx SA); enzyme inhibitors, e.g., inhibitor of tryptase such as APC-2059 (Axys Pharmaceuticals); p38 kinase inhibitors such as SB-281832 (GlaxoSmithKline); a4 integrin inhibitors such as SB 683698 (GlaxoSmithKline); protein or peptide inhibitors of TNF such as CytoAb (Protherics) and RDP58 (SangStat Corporation) antisense inhibitors of ICAM-1 such as ISIS 2302 (ISIS Pharmaceuticals); NF-kappa-B inhibitors such as P54 (Phytopharm); neurokinin-1 antagonists such as C 96348 (Pfizer); antisense inhibitors of TNF such as ISIS 104838 (ISIS Pharmaceuticals); monoclonal antibodies against TNF-α such as CDP 571 (Celltech) and infliximab (Schering Plough), monoclonal antibodies against IL-12 such as ABX-IL8 (Abgenix); monoclonal antibodies against IL-6 such as MRA (Chugai); monoclonal antibodies against CD40 such as TNX-100 (Tanox), monoclonal antibodies against a4β7 integrin receptor such as MLN-02 (Millennium); monoclonal antibodies against a4 integrin such as Natalizumab (Elan Corporation) or their functionally active fragments such as CDP 870 (Celltech); keratinocyte growth factor such as repifermin (GlaxoSmithKline); interferon (cytokin) such as IFN-b-1a (Serono); flavonoids such as DA-6034 (Dong A); glucocorticoids such as etiprednol dicloaceatate (IVAX); analogues of GLP-2 such as ALX 0600 (NPS Pharmaceuticals); small molecule glutathione peroxidase mimics such as BTX-51702 (OXIS Pharmaceuticals); small molecule phosphodiesterase IV inhibitors such as CDC-801 (Celgene); thiazole derivatives inhibiting superoxide production by human neutrophils such as OPC-6535 (Otsuka); 5-lipoxygenase inhibitors and L-selectin antagonists such as AM-24 (Cantabria); omega-3 unsaturated fatty acids such as EPA and DHA (Tillots Pharma); bactericidal/permeability-increasing (BPI) agents such as Neuprex™ (Xoma); guanyl-hydrazone compounds such as CNI-1493 (Cytokine Pharma Sciences); selective apoptotic anti-neoplastic drugs such as CP-461 (Cell Pathways); thalidomide; and recombinant human interleukin-11. The term “antibodies” includes active fragments thereof. Non-limiting examples of such compounds are listed in Table 1.

TABLE 1*

| Company | Product Name | Product Type/Comments |
|---|---|---|
| AstraZeneca | Budesonide | Synthetic steroid |
| Ferring | Mesalazine | Microsphere formulation of 5- |
| Pharmaceuticals | | aminosalicylate |
| Provalis | Mesalazine | pH-sensitive coated form of 5- |
| Procter & Gamble | | aminosalicylate |
| Pharmacia | Olsalazine | Salicylate |
| Shire Pharmaceuticals | Balsalazide | Salicylate |
| Pharmacia | Methotrexate | Immunosuppressive agent |
| Wyeth | | |
| GlaxoSmithKline | Azathioprine | Immunoactive and immunosuppressive |
| | 6-mercaptopurine | agents |
| Novartis | Cyclosporin | Immunosuppressive agent |
| Schering Plough | Infliximab | Humanized monoclonal antibody against |
| | | TNF-α |
| Dong-A | DA-6034 | Flavonoid |
| Abgenix | ABX-IL8 | Monoclonal antibody against IL-12 |
| Chugai | MRA (anti-IL-6) | Humanized monoclonal antibody against IL- |
| | | 6 for IBD |
| Nobex | Apaza | Orally-active drug targeting the lower GI |
| | | tract which combines anti-inflammatory and |
| | | immunomodulatory properties |
| Tripep | TNF-Alpha inhibitor | Protein polymerization inhibitor that inhibits |
| | | TNF-α |
| GlaxoSmithKline | SB-281832 | p38 kinase inhibitor (for IBD) |
| | SB-683698 | Anti-inflammatory inhibitor of α4 integrin |
| | | (for IBD) |
| | Repifermin | Keratinocyte growth factor (for IBD) |
| IV AX | Etiprednol dicloacetate | Orally-active glucocorticoid, rapidly |
| | (EPDC) | converted to its inactive form after |
| | | absorption. |
| NPS Pharmaceuticals | ALX-0600 | 33-amino-acid peptide analogue of |
| | | glucagon-like peptide-2 (GLP-2). |
| OXIS Pharmaceuticals | BXT-51702 | A small molecule glutathione peroxidase |
| | | mimic. Accelerates metabolism of peroxides, |
| | | is a potent inhibitor of NF-κB, prevents |
| | | oxidative damage and downregulates the |
| | | inflammatory response. |
| ISIS Pharmaceuticals | ISIS 104838 | Anti-sense TNF inhibitor |
| | ISIS 2302 | Anti-sense ICAM-1 inhibitor (for IBD) |
| NiCox SA | NiCox 456 | NO-releasing mesalazine (for IBD) |
| | NiCox 1015 | NO-releasing prednisolone derivative (for |
| | | IBD) |
| Protherics | CytoAb | Protein inhibitor of TNF |
| Pfizer | C 96348 | Antagonist of neurokinin-1 (NK-1) |
| Phytopharm plc | P54 | NF-kappa-B inhibitor (for IBD) |
| Tanox Inc | TNX-100 | Monoclonal antibody inhibitor of CD40 |
| Celgene | CDC-801 | Lead compound from a series of small, |
| | | orally-active phosphodiesterase IV inhibitors |
| | | (SelCID; Selective Cytokine Inhibitory |
| | | Drugs). |
| | Thalidomide | TNF inhibitor |
| Otsuka | OPC-6535 | Lead compound in a series of non-peptidic, |
| | | thiazole derivatives, acting as an inhibitor of |
| | | superoxide production by human neutrophils |
| SangStat Corporation | RDP-58 | Orally-active peptide inhibitor of TNFα |
| | | mRNA translation. It prevents translation of |
| | | the TNF protein rather than binding to the |
| | | protein to inhibit function. |
| Cantabria | AM-24 | 5-lipoxygenase inhibitor and L-selectin |
| | | antagonist |
| Abbott (joint | D2E7 | Human monoclonal antibody against TNFα |
| development with | | |
| Cambridge Antibody | J695 | Human monoclonal antibody against IL-12 |
| Technology plc) | | |
| Axys Pharmaceuticals | APC-2059 | Enzyme (tryptase) inhibitor (for IBD) |
| Fujisawa | FK506 (tacrolimus) | Immunosuppressive macrolide |
| Millenium | MLN-02 | Humanized monoclonal antibody to the a4β7 |
| | | integrin receptor (for IBD) |
| Oxis Pharmaceuticals | GPx | Glutathione peroxidase mimic (in UC) |
| Serono | IFN-beta-1a | Interferon (cytokine) for IBD |
| | rTBP-1 | Protein inhibitor of TNF |
| Astra Zeneca | Rofleponide | Oral steroid with topical action (for IBD) |
| Celltech | CDP 870 | 3^{rd} generation PEG humanized anti-TNFα |
| | | antibody fragment |
| | CDP 571 | Fully humanized monoclonal antibody |
| | | against TNFα |
| Alizyme | ATL-2502 | Steroid derivative in special colonic delivery |
| | | formulation |
| Elan Corporation | Natalizumab | Humanized monoclonal antibody against α4 |
| | | integrin |
| Inkine Pharmaceuticals | CBO-1011 | Steroidal molecule |
| Schering Plough | Tenovil (IL-10) | Anti-inflammatory cytokine |
| Tillotts Pharma | EPA | Enteric coated form of purified fish oil |
| | DHA | containing free fatty acid forms of |
| | | eicosapentaenoic acid (EPA) and |
| | | docosahexaenoic-omega-3 acid DHA |
| Xoma | rBPI21 (Neuprex ™) | A recombinant bactericidal/permeability- |
| | | increasing (BPI) protein. Kills gram- |
| | | negative bacteria and neutralizes the |
| | | bacterial endotoxin |
| Cytokine Pharma | CNI-1493 | A synthetic guanylhydrazone compound, |
| Sciences | | Inhibits the synthesis of inflammatory |
| | | cytokines such as TNF-α and IL-1 |
| Cell Pathways | CP-461 | A Selective Apoptotic Antineoplastic Drug |
| | | (SAAND). Induces apoptosis in neoplastic |
| | | cells by inhibiting cyclic guanosine mono- |
| | | phosphate phosphodiesterase (cGMP PDE) |
| Wyeth-Ayerst Research | rhIL-11 | A recombinant human interleukin-11. |
| | | Affects class II antigen processing and |
| | | colonic epithelial cell proliferation and |
| | | metabolism |

*Sources: Pharmaprojects, PJB Publications Ltd., status: April 2003; Target Crohn's and Colitis, Information Booklet published by the Association of the British Pharmaceutical Industry, status: February 2002); FDA at www.clinicaltrials.gov, status: April 2003).

The therapeutic and prophylactic methods, medicaments, and uses according to the invention do preferably not co-use an anti-inflammatory agent selected from the group consisting of systemically active corticosteroids. Examples of such systemically active corticosteroids include betamethasone, cortisone, hydrocortisone, methylprednisolone, prednisolone and prednisone.

Exemplary combinations of an antiviral active against a Herpes virus with other active agents against inflammatory bowel disease that can be used according to the method of this invention include one or more antivirals selected from the group consisting of antivirals active against a Herpes viruses and at least one pharmaceutically acceptable salt thereof with one or more compounds selected from list (a), and/or with one or more compounds selected from list (b), and/or with one or more compounds selected from list (c).

Preferably, the medicaments against inflammatory bowel disease according to the invention may contain a combination of an antiviral active against a Herpes virus and at least one additional active agent selected from any of the above lists (a), (b) and (c). The medicaments according to this embodiment are intended for the simultaneous, separate or successive administration of the antiviral active against a Herpes virus and the additional active agent, with the proviso that these combination medicaments do not contain an anti-inflammatory agent selected from the group consisting of salicylates and salicylate prodrugs.

In one preferred embodiment, the medicaments of this invention comprise an antiviral selected from the group consisting of acyclovir, acyclovir sodium, brivudine, cidofovir, famciclovir, foscarnet, ganciclovir, penciclovir, trifluridine, valacyclovir, valgancyclovir, and mixtures thereof.

One preferred embodiment of a combination medicament comprises an antiviral active against a Herpes virus such as famciclovir and a systemically inactive corticosteroid such as budesonide, fluticasone or pharmaceutically acceptable salts thereof. In this embodiment, the systemically inactive corticosteroid preferably comprises an enteric coating when formulated for oral administration.

Another preferred embodiment of a combination medicament comprises acyclovir sodium and infliximab.

The medicaments of this invention, whether alone or in combination with a additional active agent, can be formulated of any type of administration to a subject, including, but not limited to, intravenous, parenteral, oral, inhalation, topical, transdermal, or rectal administration, continuous infusion, or administration with an osmotic pump or a sustained release implant. When the medicaments are combination medicaments, the antiviral agent and the additional active agent can be formulated in admixture or in separate formulations for simultaneous (parallel) or separate administration.

In the embodiment where one or more agents active against inflammatory bowel diseases such as those of list (a) are co-used with at least one antiviral active against a Herpes virus or at least one pharmaceutically acceptable salt thereof or mixtures thereof, they may be co-administered in therapy and/or co-formulated in an amount or dosage which is about the same as about half the amount or dosage that, when used without the antiviral, is effective to bring about a reduction in the expression of proinflammatory cytokines (such as TNF-α, TNF-β, INF-γ, IL-2, IL-12) in the serum or in a tissue sample of the intestine mucosa of the subject to be treated.

In the embodiment wherein one or more agents active against inflammatory bowel diseases such as those of list (b) are co-used with at least one antiviral active against a Herpes virus or at least one pharmaceutically acceptable salt thereof or mixtures thereof, they may be co-administered in therapy or prevention and/or co-formulated in an amount or dosage which is about the same as about half the amount or dosage that, when used without the antiviral (in case an ordinary viral infection), is able to promote an observable (e.g. by RT-PCR) reduction in virus load and/or propagation.

In the embodiment wherein one or more active agents against inflammatory bowel disease such as those of list (c) are co-used with at least one antiviral active against a Herpes virus or at least one pharmaceutically acceptable salt thereof or mixtures thereof, they may be administered in therapy and/or be co-formulated in an amount or dosage which is about the same as about half the amount or dosage that, when used without the antiviral, is effective in promoting its respective functional effect, which effect and corresponding assaying technique is described in the respective compound's medicament information and/or drug master file.

In embodiments where at least one antiviral active against a Herpes virus or at least one pharmaceutically acceptable salt thereof or mixtures thereof is administered together in admixture, separate and simultaneously, or successively with one or more additional active agents effective against inflammatory disease, it is well within the skill of those of ordinary skill in the art to decide, depending on the type of antiviral and the type of additional active agent to be co-administered, what type of administration route to choose for each compound. It is further within the skill of one of ordinary skill in the art to determine whether the antiviral(s) and the additional active agents a can be co-formulated into one single composition or whether, for example due to some incompatibility, they should be formulated into separate dosage forms to then be used as a kit, or administered independently but concomitantly or successively according to the respective dosing regimes. Likewise the proper choice of excipients and/or diluents is within the knowledge of the skilled person, particularly as antivirals active against Herpes viruses, and the additional active agents per se are known compounds that have been previously used in other therapies and indications.

To prepare the pharmaceutical compositions according to this invention, a therapeutically or prophylactically effective amount of one or more antivirals selected from the group consisting of antivirals active against Herpes viruses and pharmaceutically acceptable salt thereof (as well as a compound provided in list (a), (b), or (c) if co-used with the antiviral) is preferably intimately admixed with a pharmaceutically acceptable carrier according to conventional pharmaceutical compounding techniques to produce a dose. A carrier may take a wide variety of forms depending on the form of preparation desired for administration, e.g., oral or parenteral. Examples include excipients such as stabilizers (to promote long term storage), emulsifiers, binding agents, thickening agents, salts, preservatives, solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the ribavirin, its use in the therapeutic compositions and preparations is contemplated. Supplementary active ingredients can also be incorporated into the compositions and preparations as described herein.

In preparing pharmaceutical compositions in oral dosage form, any of the usual pharmaceutical media may be used. Thus, for liquid oral preparations such as suspensions, elixirs and solutions, suitable carriers and additives including water, glycols, oils, alcohols, flavoring agents, preservatives, coloring agents and the like may be used. For solid oral preparations such as powders, tablets, capsules, and for solid preparations such as suppositories, suitable carriers and additives including starches, sugar carrier, such as dextrose, mannitol, lactose and related carriers, diluents, granulating agents, lubricants, binders, disintegrating agents and the like may be used. If desired, the tablets or capsules may be enteric-coated or sustained release by standard techniques.

For parenteral formulations, the carrier will usually comprise sterile water or aqueous sodium chloride solution, though other ingredients including those that aid dispersion may be included. Of course, where sterile water is to be used and maintained as sterile, the compositions and carriers must also be sterilized. Injectable suspensions may also be prepared, in which case appropriate liquid carriers, suspending agents and the like may be employed.

In the case of the combination therapies comprising co-use or co-administration of at least one antiviral active against a Herpes virus and/or at least one pharmaceutically acceptable salt thereof with an additional active agent against inflammatory bowel disease such as those of lists (a), (b), and (c), the antiviral and the additional active agent may be formulated in admixture into one single dosage. Alternatively they may be formulated in separate dosage forms for the simultaneous, separate or sequential use of the two types of dosage forms and/or to be provided as a medication kit with appropriate directions of use.

Another aspect of this invention is the use of at least one antiviral active against a Herpes virus and/or at least one pharmaceutically acceptable salt thereof for the preparation of a medicament against inflammatory bowel disease, with the proviso that the use excludes the use of anti-inflammatory agents selected from the group consisting of salicylates and salicylate prodrugs. Examples of suitable antivirals include acyclovir, acyclovir sodium, brivudine, cidofovir, famciclovir, foscarnet, ganciclovir, penciclovir, trifluridine, valacyclovir, valgancyclovir, and mixtures thereof.

In addition, this invention provides the use of one or more antivirals selected from the group consisting of antivirals active against Herpes viruses and pharmaceutically acceptable salt thereof in combination with at least one additional agent effective against inflammatory bowel disease. The additional agent includes one or more compounds selected from one or more of the following groups: (i) an agent selected from the group consisting of corticosteroids, mercaptopurine, azathioprine, metothrexate, cyclosporine and tacrolimus; and/or (ii) an antiviral active against a Herpes virus and pharmaceutically acceptable salts thereof; and /or (iii) an agent selected from the group consisting of nitric oxide releasing steroid derivatives, nitric oxide-releasing salicylates, enzyme inhibitors, p38 kinase inhibitors, a4 integrin inhibitors, protein and peptide inhibitors of TNF, antisense inhibitors of ICAM-1, NF-kappa-B inhibitors, neurokinin-1 antagonists, antisense inhibitors of TNF, monoclonal antibodies or antibody fragments against TNF-α, monoclonal antibodies or antibody fragments against IL-12, monoclonal antibodies or antibody fragments against IL-6, monoclonal antibodies or antibody fragments against CD40, monoclonal antibodies or antibody fragments against a4β7 integrin receptor, monoclonal antibodies or antibody fragments against a4 integrin, keratinocyte growth factor, interferon, flavonoids, glucocorticoids, analogues of GLP-2, small molecule glutathione peroxidase mimics, small molecule phosphodiesterase IV inhibitors, thiazole derivatives inhibiting superoxide production by human neutrophils, 5-lipoxygenase inhibitors, L-selectin antagonists, omega-3 unsaturated fatty acids, bactericidal/permeability-increasing (BPI) agents, guanyl-hydrazone compounds, selective apoptotic antineoplastic drugs, thalidomide, and recombinant human interleukin- 11.

The invention will now be illustrated in further detail by the following non-limiting examples.

### EXAMPLE 1

### Intravenous Infusion Solution

| | Content | |
|---|---|---|
| | (pers 100 ml infusion | |
| Ingredient | solution) | Function |
| acyclovir sodium | 109.8 mg | antiviral active against herpes |
| | | and/or cytomegaly |
| WFI | ad 100 mL | solvent |
| Ph. Eur. | | |

### EXAMPLE 2

### Intravenous Infusion Solutions

| | | |
|---|---|---|
| | Content | |
| | (per 100 mL | |
| | infusion | |
| Ingredient | solution) | Function |

Solution 1:

| | | |
|---|---|---|
| acyclovir sodium | 109.8 mg | antiviral active against herpes |
| | | and/or cytomegaly |
| WFI | ad 100 mL | solvent |
| Ph. Eur. | | |

Solution 2:

| | | |
|---|---|---|
| infliximab | 100 mg | immunomodulating monoclonal |
| | | antibody against TNF-α |
| WFI | ad 10 mL | solvent |
| Ph. Eur. | | |
| sodium chloride | ad 250 mL | solvent/diluent |
| intravenous infusion | | |
| (0.9% w/v)B.P. | | |

Solution 1 and Solution 2 are administered in parallel using two separate infusion lines.

### EXAMPLE 3

### Oral Medicament with Separate Dosage Forms of the Antiviral Active Against a Herpes Virus and the Additional Active Agent

| | | | |
|---|---|---|---|
| | | Active | |
| | Marketed brand | pharmaceutical | |
| Medicament | name | ingredient | Function |

Oral preparation 1:

| | | | |
|---|---|---|---|
| film-coated tablet | Famvir ® | famciclovir | antiviral active |
| | | 250 mg | against |
| | | | herpes and/or |
| | | | cytomegaly |

Oral preparation 2:

| | | | |
|---|---|---|---|
| enteric-coated | Entocort EC ® | budesonide | Systemically |
| capsule | or | 3 mg | inactive |
| | Budenofalk ® | | corticosteroid |

Oral preparation 1 and Oral preparation 2 are administered in parallel.

The foregoing description is considered as illustrative only of the principles of the invention. Further, since numerous modifications and changes will be readily apparent to those skilled in the art, it is not desired to limit the invention to the exact construction and process shown as described above. Accordingly, all suitable modifications and equivalents may be resorted to falling within the scope of the invention as defined by the claims that follow.

The words “comprise,” “comprising,” “include,” “including,” and “includes” when used in this specification and in the following claims are intended to specify the presence of stated features, integers, components, or steps, but they do not preclude the presence or addition of one or more other features, integers, components, steps, or groups thereof.

1. A method of treatment or prophylaxis of an inflammatory bowel disease in a subject in need of said treatment or prophylaxis, said method comprising:
providing at least one antiviral selected from the group consisting of antivirals active against Herpes viruses and pharmaceutically acceptable salts thereof; and
administering the antiviral to the subject in an amount effective to treat or prevent the inflammatory bowel disease, with the proviso that the method excludes administration of an anti-inflammatory agent selected from the group consisting of salicylates and salicylate prodrugs.

2. The method of claim 1, wherein the antiviral is selected from the group consisting of acyclovir, acyclovir sodium, brivudine, cidofovir, famciclovir, foscarnet, ganciclovir, penciclovir, trifluridine, valacyclovir, valgancyclovir, and mixtures thereof.

3. The method of claim 1, wherein the antiviral is acyclovir sodium.

4. The method of claim 1, further providing at least one additional agent effective against an inflammatory bowel disease, wherein the antiviral and the additional agent are administered to said subject simultaneously as an admixture, separately and simultaneously, or separately in any order.

5. The method of claim 4, wherein the additional agent is one or more agents selected from any of the following groups:
(i) an agent selected from the group consisting of corticosteroids, mercaptopurine, azathioprine, metothrexate, cyclosporine and tacrolimus;
(ii) an additional antiviral selected from the group consisting of antivirals other than antivirals active against Herpes viruses and other than pharmaceutically acceptable salts of antivirals active against Herpes viruses thereof; and
(iii) an agent selected from the group consisting of nitric oxide releasing steroid derivatives, nitric oxide-releasing salicylates, enzyme inhibitors, p38 kinase inhibitors, a4 integrin inhibitors, protein and peptide inhibitors of TNF, antisense inhibitors of ICAM-1, NF-kappa-B inhibitors, neurokinin-1 antagonists, antisense inhibitors of TNF, monoclonal antibodies or antibody fragments against TNF-α, monoclonal antibodies or antibody fragments against IL-12, monoclonal antibodies or antibody fragments against IL-6, 10 monoclonal antibodies or antibody fragments against CD40, monoclonal antibodies or antibody fragments against a4β7 integrin receptor, monoclonal antibodies or antibody fragments against a4 integrin, keratinocyte growth factor, interferon, flavonoids, glucocorticoids, analogues of GLP-2, small molecule glutathione peroxidase mimics, small molecule phosphodiesterase IV inhibitors, thiazole derivatives inhibiting superoxide production by human neutrophils, 5-lipoxygenase inhibitors, L-selectin antagonists, omega-3 unsaturated fatty acids, bactericidal/permeability-increasing (BPI) agents, guanyl-hydrazone compounds, selective apoptotic antineoplastic drugs, thalidomide, and recombinant human interleukin-11.

6. The method of claim 5, wherein the corticosteroids are systemically inactive corticosteroids.

7. The method of claim 6, wherein the corticosteroids are selected from the group consisting of budesonide, fluticasone and pharmaceutically acceptable salts thereof.

8. The method of claim 6, wherein the corticosteroids are formulated with an enteric coating.

9. The method of 5, wherein the additional antiviral of group (ii) is selected from the group consisting of abacavir, adefovir, amantadine, amprenavir, atazanavir, capravirine, delavirdine, didanosine, efavirenz, emivirin, emtricitabine, enfurvirtide, fosamprenavir, idoxuridine, indinavir, lamivudine, lopinavir, memantine, mozenavir, nelfinavir, nevirapine, oseltamivir, rimantidine, pentafuside, ritonavir, saquinavir, stavudine, tenofovir, tipranavir, zalcitabine, zanamivir, and zidovudine.

10. The method of claim 4, wherein the additional agent is infliximab.

11. The method of claim 4, wherein the antiviral active against a Herpes virus is acyclovir sodium and the additional agent is infliximab.

12. The method of claim 1 or 4, wherein the administration comprises parenteral administration, oral administration, inhalation, topical administration, transdermal administration, rectal administration, continuous infusion, or administration with an osmotic pump or a sustained release implant.

13. The method of claim 1, wherein the step of administering comprises orally administering the antiviral in a dose between about 200 mg to about 5 g per day for about 1 week to 12 months.

14. The method of claim 13, wherein the step of administering comprises orally administering the antiviral in a dose between about 200 mg to about 5 g per day for about 1 week to 4 weeks.

15. The method of claim 4, wherein the step of administering comprises orally administering to the subject for a period between about 1 week to 12 months (a) the antiviral in a dose between about 200 mg to about 5 g per day and (b) the additional agent in an amount that is between about half the dosage and the same dosage of the additional agent which, when administered alone, is effective to treat or prevent the inflammatory bowel disease.

16. The method of claim 4, wherein the step of administering comprises orally administering about 250 mg of the antiviral and about 3 mg of the additional agent.

17. The method of claim 16, wherein the antiviral and the additional agent are formulated in separate dosage forms.

18. The method of claim 17, wherein the antiviral is famciclovir and the additional agent is budesonide.

19. The method of claim 18, wherein the famciclovir and the budesonide are administered in parallel.

20. The method of claim 18, wherein the budesonide is formulated with an enteric coating.

21. The method of claim 4, wherein the step of administering comprises orally administering to the subject for a period between about 1 to 4 weeks (a) the antiviral in a dose between about 200 mg to about 5 g per day and (b) the additional agent in an amount that is between about half the dosage and the same dosage which, when the additional agent is administered alone, is effective to treat or prevent said inflammatory bowel disease.

22. The method of claim 1, wherein the step of administering comprises intravenously administering for a period between about seven to ten days an intravenous infusion of the antiviral in a dose between about 5 to about 15 mg/kg body weight of the subject over a period of 60 minutes every six to eight hours.

23. The method of claim 1, wherein the disease is selected from the group consisting of pseudomembranous colitis, hemorrhagic colitis, hemolytic-uremic syndrome colitis, collagenous colitis, ischemic colitis, radiation colitis, drug and chemically induced colitis, diversion colitis, ulcerative colitis, irritable bowel syndrome, irritable colon syndrome and Crohn's disease.

24. The method of claim 23 wherein said Crohn's disease is selected from the group consisting of active Crohn's disease, refractory Crohn's disease, and fistulizing Crohn's disease.

25. The method of claim 1, wherein the subject is a human.

26. A medicament against an inflammatory bowel disease, comprising one or more antivirals in combination with one or more additional agents active against the inflammatory bowel disease for administration to a mammal simultaneously in admixture, separately and simultaneously, or separately in any order, wherein the antiviral is in an amount effective to treat or prevent the inflammatory disease and comprises one or more antivirals selected from the group consisting of antivirals active against Herpes viruses and pharmaceutically acceptable salts thereof, with the proviso that the medicament excludes anti-inflammatory agents selected from the group consisting of salicylates and salicylate prodrugs.

27. The medicament of claim 26, wherein the antiviral is selected from the group consisting of acyclovir, acyclovir sodium, brivudine, cidofovir, famciclovir, foscarnet, ganciclovir, penciclovir, trifluridine, valacyclovir, valgancyclovir, and mixtures thereof.

28. The medicament of claim 26, wherein the antiviral is acyclovir sodium.

29. The medicament of claim 26, wherein the additional agent is one or more agents selected from any of the following groups:
(i) an agent selected from the group consisting of corticosteroids, mercaptopurine, azathioprine, metothrexate, cyclosporine and tacrolimus;
(ii) an antivirals other than antivirals active against Herpes viruses and other than pharmaceutically acceptable salts of antivirals active against Herpes viruses; and
(iii) agents selected from the group consisting of nitric oxide releasing steroid derivatives, nitric oxide-releasing salicylates, enzyme inhibitors, p38 kinase inhibitors, a4 integrin inhibitors, protein and peptide inhibitors of TNF, antisense inhibitors of ICAM-1, NF-kappa-B inhibitors, neurokinin-1 antagonists, antisense inhibitors of TNF, monoclonal antibodies or antibody fragments against TNF-α, monoclonal antibodies or antibody fragments against IL-12, monoclonal antibodies or antibody fragments against IL-6, monoclonal antibodies or antibody fragments against CD40, monoclonal antibodies or antibody fragments against a4β7 integrin receptor, monoclonal antibodies or antibody fragments against a4 integrin, keratinocyte growth factor, interferon, flavonoids, glucocorticoids, analogues of GLP-2, small molecule glutathione peroxidase mimics, small molecule phosphodiesterase IV inhibitors, thiazole derivatives inhibiting superoxide production by human neutrophils, 5-lipoxygenase inhibitors, L-selectin antagonists, omega-3 unsaturated fatty acids, bactericidal/permeability-increasing (BPI) agents, guanyl-hydrazone compounds, selective apoptotic antineoplastic drugs, thalidomide, and recombinant human interleukin-11.

30. The medicament of claim 29, wherein the additional agent is a systemically inactive corticosteroid.

31. The medicament of claim 30, wherein the corticosteroid is budesonide, fluticasone, or pharmaceutically acceptable salts thereof.

32. The medicament of claim 30, wherein the corticosteroid is formulated with an enteric coating for oral administration.

33. The medicament of claim 29, wherein the antiviral of group (ii) is selected from the group consisting of abacavir, adefovir, amantadine, amprenavir, atazanavir, capravirine, delavirdine, didanosine, efavirenz, emivirin, emtricitabine, enfurvirtide, fosamprenavir, idoxuridine, indinavir, lamivudine, lopinavir, memantine, mozenavir, nelfinavir, nevirapine, oseltamivir, rimantidine, pentafuside, ritonavir, saquinavir, stavudine, tenofovir, tipranavir, zalcitabine, zanamivir, zidovudine and pharmaceutically acceptable salts thereof.

34. The medicament of claim 29, wherein the agent is infliximab.

35. The medicament of claim 29, wherein the antiviral is acyclovir sodium and said additional agent is infliximab.

36. The medicament of claim 26 formulated for oral administration.

37. The medicament of claim 36, comprising the antiviral in an amount between about 200 mg and 5 grams.

38. The medicament of claim 26 formulated as an intravenous infusion solution.

39. The medicament of claim 38, comprising about 109.8 mg of the antiviral per 100 mL of infusion solution.

40. The medicament of claim 39, wherein said antiviral is acyclovir sodium.

41. The medicament of claim 38, comprising about 109.8 mg of the antiviral per 100 mL of infusion solution and about 100 mg of the additional agent per 100 mL of infusion solution.

42. The medicament of claim 41, wherein said antiviral is acyclovir sodium and said additional agent is infliximab.

43. The medicament of claim 38, wherein the antiviral and the additional agent are formulated in separate infusion solutions for separate but simultaneous administration.

44. The medicament of claim 36, comprising about 250 mg of the antiviral and about 3 mg of the additional agent.

45. The medicament of claim 44, wherein the antiviral and the additional agent are formulated in separate dosage forms.

46. The medicament of claim 45, wherein the antiviral is famciclovir and said additional agent is budesonide.

47. The medicament of claim 46, wherein the famciclovir and the budesonide are administered in parallel.

48. The medicament of claim 46, wherein the budesonide is formulated with an enteric coating.

49. The medicament of claim 26, wherein said medicament is formulated for intravenous administration, parenteral administration, oral administration, inhalation, topical administration, transdermal administration, rectal administration, continuous infusion, or administration with an osmotic pump or a sustained release implant.

50. The medicament of claim 26, wherein the disease is selected from the group consisting of pseudomembranous colitis, hemorrhagic colitis, hemolytic-uremic syndrome colitis, collagenous colitis, ischemic colitis, radiation colitis, drug and chemically induced colitis, diversion colitis, ulcerative colitis, irritable bowel syndrome, irritable colon syndrome and Crohn's disease.

51. The medicament of claim 51, wherein said Crohn's disease is selected from the group consisting of active Crohn's disease, refractory Crohn's disease, and fistulizing Crohn's disease.

52. Use of one or more antivirals selected from the group consisting of antivirals active against Herpes viruses and pharmaceutically acceptable salts thereof for the preparation of a medicament against inflammatory bowel disease, with the proviso that the use excludes the use of anti-inflammatory agents selected from the group consisting of salicylates and salicylate prodrugs.

53. The use according to claim 53, wherein the antiviral is selected from the group consisting of acyclovir, acyclovir sodium, brivudine, cidofovir, famciclovir, foscarnet, ganciclovir, penciclovir, trifluridine, valacyclovir, valgancyclovir, and mixtures thereof.

54. The use according to claim 53, wherein the antiviral is used in combination with at least one additional agent effective against inflammatory bowel disease selected from any of the following groups:
(i) an agent selected from the group consisting of corticosteroids, mercaptopurine, azathioprine, metothrexate, cyclosporine and tacrolimus;
(ii) an antiviral other than antivirals active against Herpes viruses and other than pharmaceutically acceptable salts of antivirals active against Herpes viruses; and
(iii) an agent selected from the group consisting of nitric oxide releasing steroid derivatives, nitric oxide-releasing salicylates, enzyme inhibitors, p38 kinase inhibitors, a4 integrin inhibitors, protein and peptide inhibitors of TNF, antisense inhibitors of ICAM-1, NF-kappa-B inhibitors, neurokinin-1 antagonists, antisense inhibitors of TNF, monoclonal antibodies or antibody fragments against TNF-α, monoclonal antibodies or antibody fragments against IL-12, monoclonal antibodies or antibody fragments against IL-6, monoclonal antibodies or antibody fragments against CD40, monoclonal antibodies or antibody fragments against a4β7 integrin receptor, monoclonal antibodies or antibody fragments against a4 integrin, keratinocyte growth factor, interferon, flavonoids, glucocorticoids, analogues of GLP-2, small molecule glutathione peroxidase mimics, small molecule phosphodiesterase IV inhibitors, thiazole derivatives inhibiting superoxide production by human neutrophils, 5-lipoxygenase inhibitors, L-selectin antagonists, omega-3 unsaturated fatty acids, bactericidal/permeability-increasing (BPI) agents, guanyl-hydrazone compounds, selective apoptotic antineoplastic drugs, thalidomide, and recombinant human interleukin-11.
